(19) United States
(12) Patent Application Publication
(10) Pub. No.: US 2005/0187527 A1
(43) Pub. Date: Aug. 25, 2005

(54) Electro-conductive textiles having enhanced uniformity of electrical resistance and heat profile and process of making same

(76) Inventor: Robert Rix, Southport (GB)

Correspondence Address: Michael G. Cameron;Jackson Walker LLP, Suite 600, 2435 North Central Expressway, Richardson, TX, 75080, US

(21) Appl. No.: 11/024,566

(22) Filed: Dec. 29, 2004

Related U.S. Application Data
(63) Continuation-in-part of application No. 10/494,852, filed on Oct. 14, 2004.

(51) Int. Cl. A61M 35/00

(52) U.S. Cl. 604/289

(57) ABSTRACT

A process (300) and resultant product, of producing a material with substantially isotropic resistive characteristics comprising carbonizing a precursor material while it is in a relaxed condition is disclosed. The precursor material preferably comprises a polymer (301) fabric. The result of the process (300) is an electro-conductive textile product (302).

### CROSS REFERENCE TO RELATED APPLICATIONS; CLAIMS OF PRIORITY

This application is a continuation-in-part of U.S. patent application Ser. No. 10/494,852, filed Nov. 1, 2002, claiming a priority date of Nov. 6, 2001, entitled “Heated Wound Dressing”; U.S. patent application Ser. No. 10/289,500, filed Nov. 5, 2002, claiming a priority date of Nov. 6, 2001 entitled “Heated Transportation Box” and U.S. patent application Ser. No. 10/475,579, filed Apr. 12, 2004, claiming a priority date of Apr. 27, 2001, entitled “Electro-conductive Textile Sensor”, the entire contents of which are incorporated herein by this reference. The Applicant hereby claim the benefits of these pending patent applications under 35 U.S.C. Section 119(e).

### FIELD OF THE INVENTION

The present invention relates generally to improved electro-conductive materials or textiles used, among other things, as heating elements or sensors, and the process of making same. More specifically, the present invention relates to the provision of, and process of making, improved electrically conductive materials which are in sheet or web form.

### BACKGROUND OF THE INVENTION

Reference is made to U.S. Pat. No. 6,172,344 B1 issued Jan. 9, 2001 to Rix, Gordon and Gerrard (the “344 Patent”), relating to the composition and manufacture of certain types of electro-conductive textiles that can be used as heating element components. Background information related to electro-conductive textiles can be found in the “344 Patent. A variety of terms are used herein to refer to the electro-conductive textile, including ECT, carbonized fabric and electro-conductive material, and precursors thereof, including woven, non-woven and knitted material, cloth and the like. For purposes hereof, such terms may be collectively referred to as ECT. Thus, as the context requires, ECT refers to the final product, as well as the precursor products that become the final electro-conductive textile or material.

### SUMMARY OF THE INVENTION

The present invention relates generally to the fabrication of electro-conductive materials or textiles, and the use thereof, among other things, as heating elements or sensors, or in a variety of applications.

### DESCRIPTION OF THE DRAWINGS

The features of the present invention will be more clearly understood from consideration of the following description in connection with accompanying drawings in which:

FIG. 1 is a first set of tables setting forth the results of testing six, 4 inch squares of ECT made in a conventional manner;

FIG. 2 is a second set of tables setting forth the results of testing six, 4 inch squares of ECT made with the improved method described herein; and

FIG. 3 is a block diagram of the improved process of making the improved ECT.

### DETAILED DESCRIPTION OF ILLUSTRATIVE EMBODIMENTS

While the manufacture and use of ECT has previously been disclosed in the '344 Patent, an improved ECT can be manufactured by using a novel process employing an alternate apparatus as disclosed herein. By using such improved process, ECT with enhanced levels of uniformity in electrical resistance can be achieved. Enhanced uniformity of electrical resistance leads to improved levels of uniformity of heat profile when an electrical potential is applied across the ECT. It is well known in the art that when a potential is applied across a resistive element, electrical energy is converted to heat energy. When a potential is applied across the improved ECT of the present invention, heat is disposed over the entire surface of the ECT substantially isotropically.

To better understand the advantages of, and significant improvements that, the present invention has over conventional ECT, reference to the method of making conventional ECT is provided below.

A conventional process of manufacturing ECT requires the pre-prepared woven material being first folded longitudinally to reduce the width sufficiently to allow entry into a carbonization oven. The folded cloth is then transported through the oven by means of a conveyor belt. To maintain progress and take up the cloth as it exits the oven, an arrangement of feed and collection rollers are located at the end of the oven. The cloth is effectively pulled through the oven. The rollers regulate the speed of the cloth through the oven. The cloth, during its horizontal passage through the oven, is in a relaxed state in the weft direction and in a restrained condition in the warp direction due to the action of the feed and collection rollers. In this method of manufacture, the still hot cloth is rolled upon exiting the oven. It is known in the art that restraint and relaxation of the cloth during the carbonization process affects the electrical resistance of the finished cloth. However, until the present invention, it has not been known how and to what degree relaxation and/or restraint impacts the isotropicity of electrical resistance of the finished cloth.

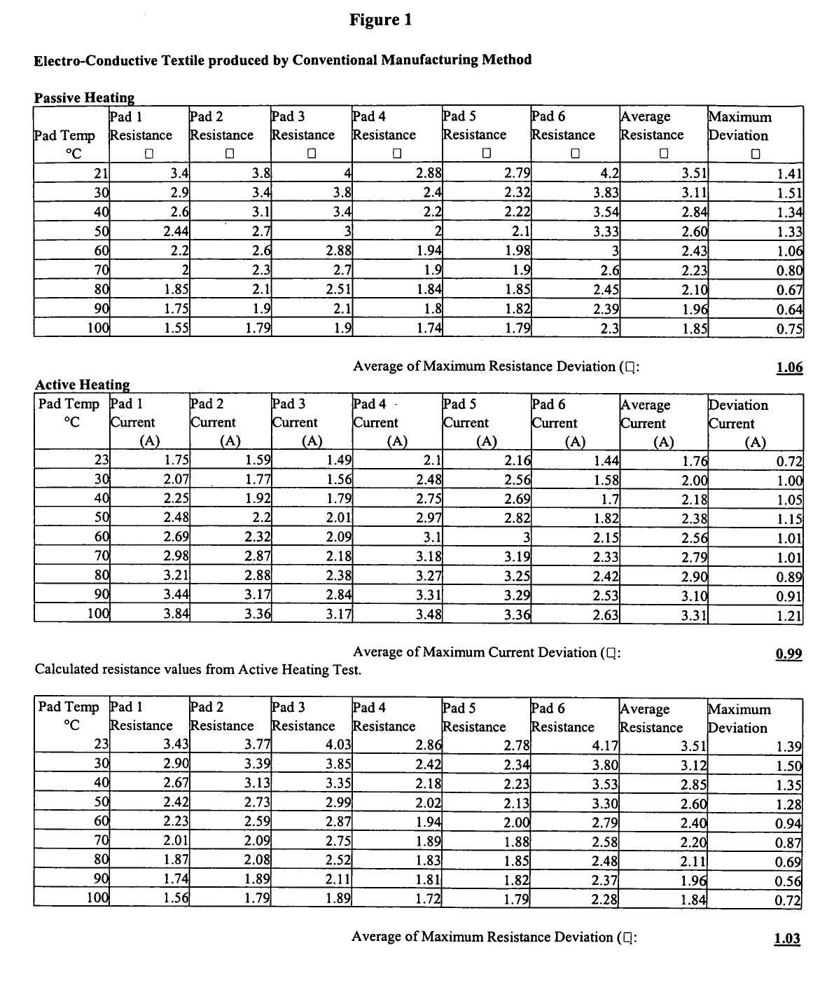

The cloth produced in the conventional manner billows or bags in the middle of the roll width, while the selvedge edge of the cloth remains taught. The line upon which the cloth is folded longitudinally prior to carbonization remains creased despite efforts to render flatness to the cloth. As a result, the electrical resistance levels of the cloth are of an endotropic nature. As a result, and as seen in the tables of FIG. 1, the heat output profile of heater elements manufactured from such cloth also present endothermic characteristics.

An improved process of processing the cloth that becomes the ECT overcomes the cited disadvantages of the conventional process. The improved manufacturing process involves the pre-preparation of sufficiently narrow woven material being transported through an oven in an unfolded and flat state. The carbonization chamber of the oven is arranged in a vertical plane. Entry to the oven is by means of a top feed roller that takes the cloth initially up to the top mouth of the oven and then controls the speed of the downward progress of the cloth in a vertical manner and in a completely relaxed and unrestrained condition. As the cloth exists the oven it is collected in a catch basket where it is allowed to settle and cool before being assembled onto rolls.

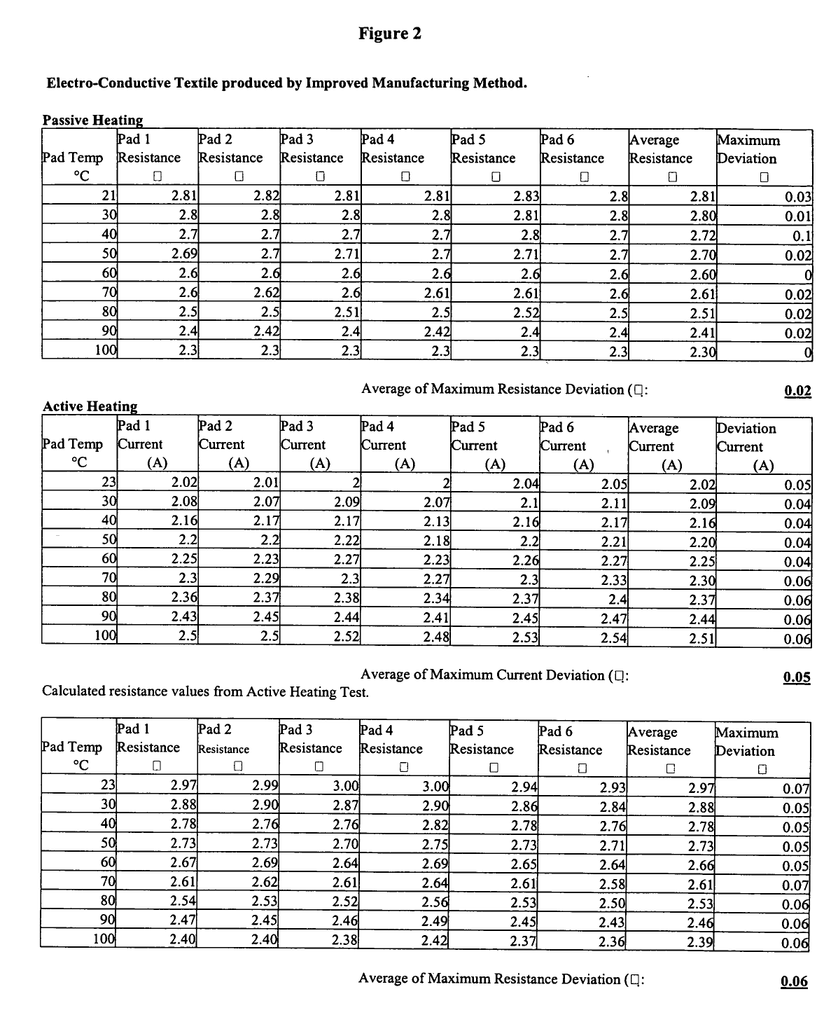

The cloth produced by the improved method lies flat and does not significantly billow or bag in the middle of the roll width. Furthermore the selvedge edge of the cloth is relaxed. The electrical resistance levels of cloth manufactured by this improved method are of an isotropic nature. As a result, and as seen in the sets of tables comprising FIG. 2, the heat output profile of heater elements manufactured from cloth made from this improved process also present isothermal characteristics.

To illustrate more specifically the results of the improved process of making ECT of the present invention, the following parameters are provided. The specifications of the conventional ECT prior to carbonization are as follows: width, 84 inches; ends per inch, 30 nominal; picks per inch, 22 nominal; finished fabric weight, 270 g/m² nominal. The specifications of the conventional ECT after carbonization are as follows: width, 67 inches nominal; finished Fabric weight 240 g/m² nominal.

The specifications of the improved ECT prior to carbonization are as follows: width, 58 inches; ends per inch, 30 nominal; picks per inch, 22 nominal; finished fabric weight, 330 g/m² nominal. The specifications of the improved ECT after carbonization are as follows: width, 48 inches; finished fabric weight 210 g/m² nominal.

As can be seen, a degree of loss of length of cloth takes place due to the unrestrained passage of the cloth through the oven. However, the improved uniformity of electrical resistance compensates for the reduced cloth length.

Referring now to FIG. 1, a set of first tables is presented showing the results of testing ECT made in the conventional manner. As seen therein, six, 4 inch squares of traditionally manufactured ECT were cut from locations equally spaced across the width of the finished cloth. These squares were incorporated into heater pads. The heater pads were heated both actively and passively to a temperature of 100° C. The electrical resistance of the heater pads was taken at the ambient temperature of 21° C. and thereafter every level 10° C. increment to 100° C. The passive heating took place in a custom made calibrated heating chamber. Temperature was measured by a K type thermocouple and recorded on a Pico TC 08 data logger. Resistance was measured with a Fluke 70 series II Multimeter.

For the active heating test, the heater pads were connected to a bench power supply TTi TSX 3510 and a 6 volt potential applied. The current draw was noted every 10° C. The electrical resistance of each heater pad was thereafter calculated using Ohms law. The heater pads under test were covered with 3 inch thick closed cell foam insulation to limit the effects of varying air currents.

Referring now to FIG. 2, a second set of tables setting forth the results of testing six, 4 inch squares of improved ECT made with the improved process described herein is provided. The tests of these samples were identical to the tests performed on the conventional ECT samples. As seen in FIG. 2, the tests took place using six, 4 inch heater pads cut from a roll of ECT manufactured by the improved process.

The isothermal characteristic of the improved ECT improves the performance of all manufactured items that employ the improved ECT as a form of a heating element or sensor within their construction.

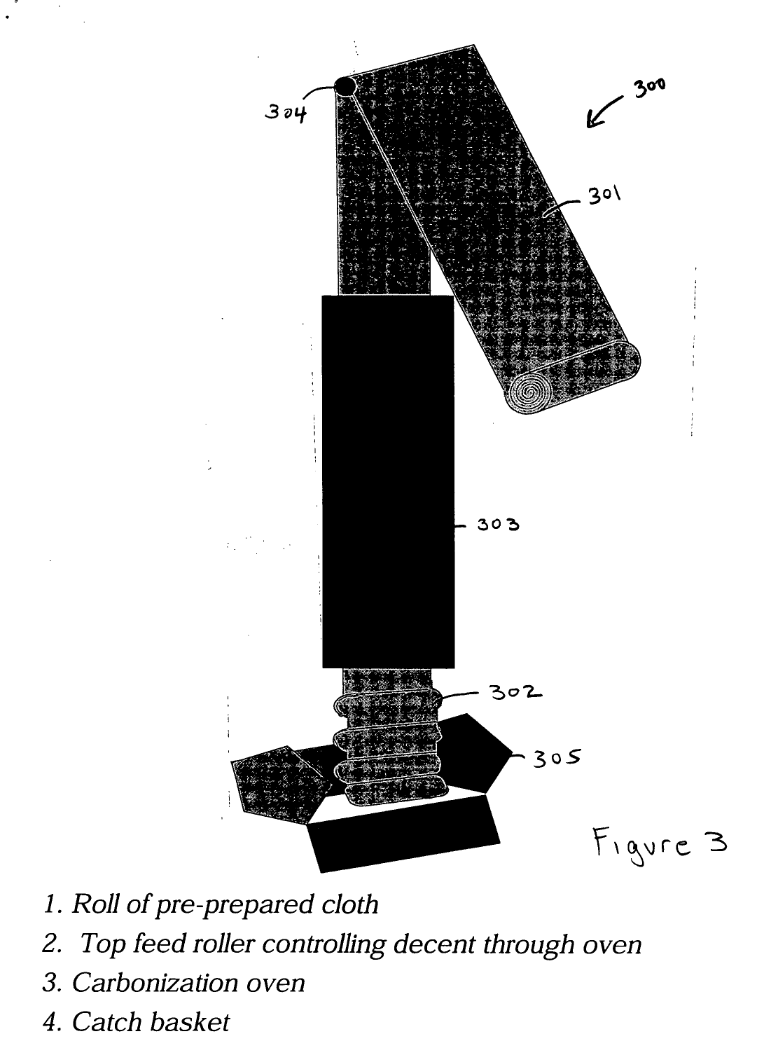

FIG. 3 provides a block diagram of an exemplary embodiment of the manufacturing process 300 of the improved ECT. In this arrangement, a process 300 of producing a material with substantially isotropic resistive characteristics comprising carbonizing a precursor material 301 while it is in a relaxed condition is implemented. The precursor material 301 preferably comprises a polymer, such as one from the group consisting of polyacrilonitrile, rayon and viscose fabric. The result of the process is an electro-conductive textile product 302. The process 300 can also be referred to as carbonizing a precursor material 301 while it is in a vertically supported position; carbonizing a precursor material 301 while it is in a vertically unrestrained position; and carbonizing a precursor material 301 while it is in a flat and unfolded state. The selvedge edge of the carbonized material 302 is allowed to relax during carbonization. This carbonized material 302 is allowed to settle and cool after carbonization. In an alternative method of describing the steps, a woven material is pre-prepared. The woven material is then fed into a carbonization chamber of a vertically arranged oven 303 by a feed mechanism 304 at the upper end of the vertically arranged oven. The woven material is transported in a regulated manner in an unfolded and flat state vertically through the carbonization chamber of the vertically arranged oven 303, whereby the woven material is converted into the carbonized material 302. In an exemplary embodiment of the process, the woven fabric before carbonizing weighs 330 grams per square meter. In such exemplary process, the woven fabric is carbonized down to 210 grams per square meter. Typically, such finished fabric weight after carbonization is about 220 grams plus or minus 10 grams. In this exemplary process, the oven temperature is between about 930 and 1050 degrees Centigrade in an atmosphere from the group consisting of Nitrogen and Argon. The speed through the machine/oven can be 9.5 meters per hour, 9 meters per hour or 8.5 meters per hour. Singly or multiple widths of cloth can be put through the oven side by side. The width is not restricted to a single or a folded width of cloth.

In one embodiment of the present invention, the carbonized material is then collected in a catch basket 305 at the lower end of the vertically arranged oven 303. After cooling, the carbonized material 302 is collected into rolls. However, there can be certain circumstances where the cloth is not collected into a catch basket to settle and cool but is allowed to drape down over transit rollers to cool and settle before being committed to final rolling onto tubes for shipment. This is done in a relaxed state. The improved ECT made by the foregoing process 300 can be used to develop heat uniformly across its surface by applying a potential difference across the carbonized material. Thus, the improved ECT can be used as a heating element of a heating system.

An electrical circuit, such as a circuit for generating a potential and sensing current can be used in conjunction with the circuit for applying and regulating a potential difference across the heating element. Such an electrical control circuit can be arranged to control the temperature of the heating element, and to derive a control signal from an electrical current passing through the heating element. Electrodes can be connected to the heating element at spaced locations enabling the application of the potential difference across the area of the carbonized material between the electrodes. The heating system incorporating the carbonized material can be configured in a variety of orientations. Such a heating element can have a protective layer on at least one side thereof. Further, the element can include a pair of opposite sides that further comprises a pair of protective layers, the protective layers each being applied to a respective one of the opposite sides. The protective layers can be arranged so as to cooperate with at least one edging strip to encapsulate the carbonized fabric heating element.

The circuit used to apply the potential difference can include at least two conductive bus bars. These bus bars, preferably, are sewn in place. Other methods of attachment, such as gluing can also be used. These bars can each comprise at least one of copper, electrically conductive metal foil, woven wire braid, woven wire strips, an electrically conductive plastics material, and conductive wires. The circuit used to regulate the voltage supplied or current drawn across the carbonized material hence can be used to control the temperature of the carbonized material or heating element.

In order to practice the improved process for producing an electrically conductive material with substantially isotropic resistive characteristics, a novel apparatus for implementing same is required. Such an apparatus for making an electrically conductive material comprises an oven with a carbonization chamber arranged in a vertical plane; a feed roller adapted to feed the material in a regulated manner to the upper end of the vertically arranged oven; the feed roller adapted to transport the material in an unfolded and flat state vertically through the carbonization chamber of the vertically arranged oven; and a catch basket at the lower end of the vertically arranged oven adapted to collect the carbonized material or cloth. Such an apparatus would be adapted to carbonize polymers, including those from the group consisting of polyacrilonitrile, rayon and viscose fabric. For example, but not as a limitation of the potential applications in which the ECT can be used, there are disclosed below a variety of items in which the improved ECT can be incorporated.

The improved ECT product of the present invention can be incorporated into a form of heated carrying bag used by seaborne rescue, ambulance, paramedic, accident and emergency crews. The heated bag could contain at least one bag of transfusible liquid and a sterile blood administration set for use in the transfusion blood, blood derivatives, saline, glucose or other tranfusible liquids. Such a heated bag incorporating the improved ECT could be powered by a battery pack. This battery pack can be contained in the base of the bag, when required for portable use, for example, at the scene of an accident. Alternatively, the heated bag incorporating the improved ECT can be powered from the electrical system of the transportation vehicle on its way to the accident scene. In such an embodiment, the ECT heating element is preferably arranged on the inner face of the back and/or sides of the heated bag. The heated bag could further have an easy open zip fastener or alternatively, a means of closure such as a hook and loop type closure system, allowing access to the heated contents. Furthermore, such a heated bag could incorporate, among other things, a reflective insulated lining for heat retention, a weatherproof flap, a rigid loop for transportation on the belt of a paramedic and a carry handle on the top and/or sides thereof.

In addition, the improved ECT product can be incorporated into, or used as a form of, a heated blanket to be used in lifeboats, and by coastguards, helicopter, air-sea rescue services, ambulance, paramedic and accident and emergency crews. Such a heated blanket could contain a heated section for warming a patient, for example, at the scene of an accident. The blanket further could have a waterproof outer surface manufactured from high visibility material and could be overlaid with light reflective strips. The heated section of such a blanket is powered by a battery pack contained in a carry pack that could be either hand carried or suspended beneath a gurney, for example, when being carried to the scene of the accident. Alternately, such a blanket could be powered from the electrical system of a transportation vehicle. Such a heated blanket would be stowed when not in use, in, for example, a high visibility transportation tube that could contain a secondary battery pack that pre-heats the blanket, for example, when en-route to the scene of the accident. The blanket could also be coupled with, or stored in a distress signaling device. Such a device can be configured as a telescopic tube that can be extended as a baton to summon help. Such a signaling system, in combination with the heated blanket, could be combined as a vehicle emergency kit.

The improved ECT product would also be useful in beds, mattresses, blankets, duvets, covers, and throws for bedding, cushions and pillows, for healthcare and domestic use. Furthermore, a heated wound dressing could benefit from the use or inclusion of a heating element constructed from the improved ECT. Many other diverse healthcare products would also benefit from the use of the improved ECT. These include heated bandages, heated plaster-casts for the treatment of broken limbs; heated wraps, splints, supports and belts used for the relief and treatment of muscular and skeletal pain and disorders. Additional uses include as a form of heated cover or wrap to be displaced over a patient during an operation or medical procedure. The blanket would have flap sections that could be moved and lifted to allow access by medical staff during medical procedures or operations. The blanket could be laminated within an anti-bacterial cover material to aid with sterilization issues.

The improved ECT product would also be useful in beds, blankets, covers for bedding, cushions and pillows. The improved ECT could also be incorporated into baby incubators, stretchers, gurneys and operating tables. Any medical device that would require or give the patient benefit from the application of heat, e.g., where heat is to be applied to or close to human tissue, could be improved by the employment of a heating element comprising the improved ECT.

Additional, less sophisticated products, would benefit from the use or incorporation of the present invention. These include articles of clothing, such as, but not limited to coats, jackets, vests, waistcoats, trousers, gloves, footwear and headgear. Such products are not limited to those that are worn by humans. The present invention could be incorporated into veterinary products such as wraps, blankets, rugs and jackets. The improved ECT of the present invention could also be used in molding tools and covers used in the composites industry and plastics forming industry where strong reliance on uniformity of heat is required. In addition, the improved ECT of the present invention can be used in covers, wraps and bags for thermally sensitive equipment such as diagnostic equipment, computers, cameras, navigational aids, similarly packages and containers used for the transportation of thermally sensitive products such as pharmaceutical products and medicines. There are a variety of uses for the improved ECT of the present invention in the automotive and vehicle manufacturing industry, including car seat heater systems, heated roof liners and door panels, heated bunks in recreational vehicles and in the sleeper section of semi-rig trailers.

There are a variety of uses and applications of the improved ECT of the present invention in the food preparation, delivery and service industry, such as heated shelves, trays, warmers and trolleys along with pizza bags. There are also a variety of uses of the improved ECT of the present invention in the construction and building industry where such improved ECT could be employed in and beneath concrete to impart warming and activation of color changing pigments and dyes used for decorative purposes. In addition, the improved ECT could be used in under floor heating systems and heated wall coverings, heated roof shingles for ice and snow thawing.

The improved ECT of the present invention can also be used in infrared and heat signature weaponry targeting devices where an even and uniform heat profile is required, such devices being used for both mobile tank and static artillery training purposes, and also to mimic the heat signature if equipment or personnel to act as a military decoy. Additionally, the improved ECT of the present invention can be used in airborne and seaborne devices for use in connection with equipment which is sensitive to discerning the heat signature of a shape or surface. For example, a target made of the improved ECT material could be shot at with artillery or ballistic fire and still function when pierced or perforated by the projectile.

The improved ECT of the present invention can also be used in conjunction with shape change materials and alloys as a thermal triggering stimulus to initiate change or to return said shape memory material or alloy to its original shape. For example, the improved ECT can be used in clothing to change its shape, look or feel thereof. In addition, it can be used in furniture that, through the application of heat to a part of its construction or surface, is adapted to be changed to an alternate and possibly more comfortable shape or position. In these type of applications of the improved ECT, the improved material could be laminated, encased or encapsulated within a variety of such shape memory materials.

The innovative teachings of the present invention are described with particular reference to the exemplary embodiments described herein. It should be understood and appreciated by those skilled in the art that the embodiments described herein provide only a few examples of the many advantageous uses and innovative teachings herein. Various alterations, modifications and substitutions can be made to the process of the disclosed invention, and the resultant product, without departing in any way from the spirit and scope of the invention.

1. A process of producing a material with substantially isotropic resistive characteristics comprising carbonizing a precursor material while it is in a relaxed condition.

2. The process of claim 1, wherein the precursor material comprises a polymer fabric.

3. The process of claim 2, wherein the polymer is one from the group consisting of polyacrilonitrile, rayon and viscose.

4. The process of claim 1, whereby the carbonizing process further comprises feeding the precursor material into an oven having an oven temperature of between about 930 and 1050 degrees Centigrade in an atmosphere of one from the group consisting of Nitrogen and Argon.

5. The process of claim 4, further comprising feeding the precursor material into the oven at a rate of between 8 meters per hour and 10 meters per hour.

6. The process of claim 5, wherein singly or multiple widths of cloth are feed into the oven side by side.

7. An electro-conductive textile product made by the process of claim 1.

8. A process of producing a material with substantially isotropic resistive characteristics, comprising carbonizing a precursor material while it is in a vertically suspended position.

9. The process of claim 8, wherein the precursor material comprises a polymer fabric.

10. The process of claim 9, wherein the polymer is one from the group consisting of polyacrilonitrate, rayon and viscose.

11. The process of claim 8, whereby the carbonizing process further comprises feeding the precursor material into an oven having an oven temperature of between about 930 and 1050 degrees Centigrade in an atmosphere consisting of one from the group of Nitrogen and Argon.

12. The process of claim 11, further comprising feeding the precursor material into the oven at a rate of between 8 meters per hour and 10 meters per hour.

13. The process of claim 12, wherein singly or multiple widths of cloth are fed into the oven side by side.

14. An electro-conductive textile product made by the process of claim 8.

15. A process of producing a material with substantially isotropic resistive characteristics comprising carbonizing a precursor material while it is in a vertically unrestrained position.

16. The process of claim 15, wherein the precursor material comprises a polymer fabric.

17. The process of claim 16, wherein the polymer is one from the group consisting of polyacrilonitrate, rayon and viscose.

18. The process of claim 15, whereby the carbonization process further comprises feeding the precursor material into an oven having an oven temperature of between about 930 and 1050 degrees Centigrade in an atmosphere from the group consisting of Nitrogen and Argon.

19. The process of claim 18, further comprising feeding the precursor material into the oven at a rate of between 8 meters per hour and 10 meters per hour.

20. The process of claim 19, wherein singly or multiple widths of cloth are feed into the oven side by side.

21. An electro-conductive textile product made by the process of claim 15.

22. A process of producing a material with substantially isotropic resistive characteristics, comprising carbonizing a precursor material while it is in a flat and unfolded state.

23. The process of claim 22, wherein the precursor material comprises a polymer woven fabric.

24. The process of claim 23, wherein the polymer is one from the group consisting of polyacrilonitrate, rayon and viscose.

25. The process of claim 22, whereby the carbonization process further comprises feeding the precursor material into an oven having an oven temperature of between about 930 and 1050 degrees Centigrade in an atmosphere from the group consisting of Nitrogen and Argon.

26. The process of claim 25, further comprising feeding the precursor material into the oven at a rat of between 8 meters per hour and 10 meters per hour.

27. The process of claim 26, wherein singly or multiple widths of cloth are feed into the oven side by side.

28. An electro-conductive textile product made by the process of claim 22.

29. The process of claim 22, further comprising the carbonized material being allowed to settle after carbonization.

30. The process of claim 29, further comprising the carbonized material being allowed to cool after carbonization.

31. The process of claim 22, wherein the selvedge edge of the carbonized material is allowed to relax during carbonization.

32. An electro-conductive textile product made by the process of claim 22.

33. The process of claim 22, wherein the cloth is allowed to drape down in a relaxed manner over transit rollers to cool and settle before being rolled onto tubes.

34. An electro-conductive textile product made by the process of claim 33.

35. A process for making an electrically conductive material, comprising:
pre-preparing a precursor material;
feeding the precursor material to a feed mechanism at the upper end of a vertically arranged oven;
transporting the precursor material in a regulated manner in an unfolded and flat state vertically through the carbonization chamber of the vertically arranged oven;
carbonizing the precursor material into carbonized material; and
collecting the carbonized material in a catch basket at the lower end the vertically arranged oven.

36. The process of claim 35, whereby the carbonizing process further comprises feeding the precursor material into the vertically arranged oven with an oven temperature of about 930 to 1050 degrees Centigrade in an atmosphere from the group consisting of Nitrogen or Argon.

37. The process of claim 36, further comprising feeding the precursor material into the oven at a rate of between 8 meters per hour and 10 meters per hour.

38. The process of claim 37, wherein singly or multiple widths of precursor material are feed into the oven side by side.

39. The process of claim 35, further comprising allowing the carbonized material to settle and cool; and
assembling the carbonized material into rolls.

40. The process of claim 35, wherein the pre-preparation of the precursor material comprises weaving a polymer fabric.

41. The process of claim 40, wherein the polymer comprises one from the group consisting of polyacrilonitrile, rayon and viscose.

42. An electro-conductive textile product made by the process of claim 35.

43. The electro-conductive textile product of claim 42, further comprising a circuit and power source for applying and regulating a potential difference across the carbonized material.

44. The electro-conductive textile product of claim 43, for use as a heating system.

45. The electro-conductive textile product of claim 43, further comprising:
the carbonized material comprising a heating element; and
a means for applying and regulating a potential difference across the heating element.

46. The electro-conductive textile product of claim 45, further comprising an electrical control circuit arranged to control the temperature of the heating element.

47. The electro-conductive textile product of claim 46, wherein the means for applying a potential difference across the heating element are electrodes connected to the heating element at spaced locations enabling the application of the potential difference across the area of the carbonized material between the electrodes.

48. The electro-conductive textile product of claim 43, formed as a heated carrying bag.

49. The product of claim 48, for use by air-sea rescue services, ambulance, paramedic and emergency crews.

50. The product of claim 48, adapted to contain at least one bag of transfusible liquid.

51. The product of claim 50, for use in transport of transfusion blood, blood derivatives, saline, glucose or other tranfusible liquids.

52. The product of claim 48, wherein such carrying bag is powered by a battery pack.

53. The product of claim 48, wherein the carrying bag is powered by the vehicle electrical system of a vehicle.

54. The product of claim 48, wherein the electro-conductive textile is used as a heating element arranged on the inner face of the back and/or sides of the carrying bag.

55. The product of claim 48, further comprising the carrying bag having an easy open zip fastener adapted to allow access to the heated contents.

56. The product of claim 48, wherein the carrying bag has fastener comprising a hook and loop type fastener.

57. The product of claim 48, further comprising the carrying bag having a reflective insulated lining for heat retention.

58. The product of claim 48, further comprising the carrying bag having a weatherproof flap.

59. The product of claim 48, further comprising the carrying bag having a rigid loop for transportation on the belt of a user.

60. The product of claim 48, further comprising the carrying bag having a carry handle on the top and/or sides thereof.

61. The electro-conductive textile product of claim 43, formed as a heated blanket.

62. The product of claim 61, further comprising a heated section thereof for warming a patient.

63. The product of claim 61, further comprising a waterproof outer surface manufactured from high visibility material.

64. The product of claim 61, further comprising being overlaid with light reflective strips.

65. The product of claim 61, further comprising a heated section of such blanket powered by a battery pack.

66. The product of claim 65, the battery pack further comprising a pack being adapted to be either hand carried or suspended beneath a gurney.

67. The product of claim 61, further comprising being powered from a vehicle battery.

68. The product of claim 61, further comprising being powered from an AC or DC source.

69. The product of claim 61, adapted to be stored in a high visibility transportation tube.

70. The product of claim 69, in combination with a distress signaling device.

71. The product of claim 70, wherein the distress signaling device is configured as a telescopic tube, adapted to be extended and waved as a baton.

72. The product of claim 71, for use as a vehicle emergency kit.

73. The product of claim 69, wherein such transportation tube incorporates a secondary battery pack adapted to pre-heat the blanket.

74. The electro-conductive textile product of claim 43, being formed as a heating element for use in or as a heated wound dressing.

75. The electro-conductive textile product of claim 43, being formed as a heating element for use in as a heated bandage.

76. The electro-conductive textile product of claim 43, being formed as a heating element for use in or as a heated plaster-cast for the treatment of a broken limb.

77. The electro-conductive textile product of claim 43, being formed as a heating element for use in a heated wrap.

78. The electro-conductive textile product of claim 43, being formed as a heating element for use in a splint.

79. The electro-conductive textile product of claim 43, being formed as a heating element for use in supports and belts used for the relief and treatment of muscular and skeletal pain and disorders.

80. The electro-conductive textile product of claim 43, being formed as a heating element for use in a heated bed.

81. The electro-conductive textile product of claim 43, being formed as a heating element for use in a heated blanket.

82. The electro-conductive textile product of claim 43, being formed as a heating element for use in a bed covering.

83. The electro-conductive textile product of claim 43, being formed as a heating element for use in a cushion or pillow.

84. The electro-conductive textile product of claim 43, being formed as a heating element for use in a baby incubator.

85. The electro-conductive textile product of claim 43, being formed as a heating element for use on a stretcher or gurney.

86. The electro-conductive textile product of claim 43, being formed as a heating element for use on an operating table.

87. The electro-conductive textile product of claim 43, being formed as a heating element for use in an articles of clothing from the group consisting of a coat, jacket, trousers, vests, waistcoats, gloves, footwear and headgear.

88. The electro-conductive textile product of claim 43, being formed as a heating element for use in a veterinary product.

89. The electro-conductive textile product of claim 43, being formed as a heating element for use in a molding tool or cover.

90. The electro-conductive textile product of claim 43, being formed as a heating element incorporated into a cover, wrap or bag used for thermally sensitive equipment.

91. The product of claim 90, wherein the thermally sensitive equipment comprises one from the group consisting of diagnostic equipment, computer, camera, and navigational aid.

92. The electro-conductive textile product of claim 43, being formed as a heating element incorporated into a package or container used for the transportation of thermally sensitive products.

93. The product of claim 92, wherein the thermally sensitive product comprises one from the group consisting of pharmaceutical products and medicines.

94. The electro-conductive textile product of claim 43, being formed as a heating element incorporated into a component of a vehicle.

95. The product of claim 94, wherein the component comprises one from the group consisting of a car seat heater system, heated roof liner, door panel, heated bunk in recreational vehicle and the sleeper section of semi-rig trailer.

96. The electro-conductive textile product of claim 43, being formed as a heating element incorporated a device used in the food preparation, delivery and service industry.

97. The product of claim 96, wherein such device comprises one from the group consisting of heated shelves, trays, warmers, trolleys and pizza delivery bags.

98. The electro-conductive textile product of claim 43, being formed as a heating element for use in the construction and building industry.

99. The product of claim 98, wherein the heating element is used for an application from the group consisting of heating concrete, under floor heating systems, heated wall coverings, and heated roof shingles for ice and snow thawing.

100. The electro-conductive textile product of claim 43, being formed as a heating element for use in infrared and heat signature weaponry targeting devices and military decoys.

101. The electro-conductive textile product of claim 43, being formed as a heating element for use in mobile tank and static artillery target training purposes.

102. The electro-conductive textile product of claim 43, being formed as a heating element for use in airborne and seaborne devices to test equipment sensitive to discerning the heat signature of a shape or surface.

103. The product of claim 102, adapted to continue to function as a heat signature when pierced or perforated by a projectile.

104. The electro-conductive textile product of claim 43, being formed as a heating element used in conjunction with shape memory materials and alloys.

105. The product of claim 104, wherein the heating element is laminated, encased or encapsulated within a variety of such shape memory materials.

106. The product of claim 104, for use in clothing to change the shape, look or feel thereof.

107. The product of claim 104, for use in furniture which is adapted to change its shape upon the application of heat.

108. The electro-conductive textile product of claim 43, for use as a heating system wherein the carbonized material has a protective layer on at least one side thereof.

109. The heating system of claim 108, wherein the carbonized material includes a pair of opposite sides and further comprises a pair of protective layers, the protective layers each being applied to a respective one of the opposite sides.

110. The heating system of claim 109, wherein the protective layers cooperate with at least one edging strip to encapsulate the carbonized material.

111. The heating system of claim 108, wherein the circuit for applying the potential difference comprise at least two conductive bus bars.

112. The heating system of claim 111, wherein said bus bars each comprise at least one from the group consisting of copper, electrically conductive metal foil, woven wire braid, woven wire strips, an electrically conductive plastics material, and conductive wires.

113. The heating system of claim 111, wherein the bus bars are sewn to the carbonized material.

114. The electro-conductive textile of claim 42, for use as a sensor.

115. A process for producing an electrically conductive material with substantially isotropic resistive characteristics, comprising:
processing a precursor cloth such that it lies flat and does not significantly billow or bag in the middle of the roll width; and
relaxing the selvedge edge of the cloth during carbonization.
